(19) United States
(12) Patent Application Publication
(10) Pub. No.: US 2021/0330279 A1
(43) Pub. Date: Oct. 28, 2021

(54) Audio Output as Half Duplex Biometric Detection Device

(71) Applicant: ESS Technology, Inc., San Jose, CA (US)

(72) Inventors: Dustin Dale Forman, Kelowna (CA); Christian Leth Petersen, Burnaby (CA); A. Martin Mallinson, Kelowna (CA)

(21) Appl. No.: 17/241,417

(22) Filed: Apr. 27, 2021

Related U.S. Application Data
(60) Provisional application No. 63/016,012, filed on Apr. 27, 2020.

(51) Int. Cl. A61B 7/04; G10L 25/51; H04R 3/04; H04R 29/00; H03F 3/183

(52) U.S. Cl. CPC A61B 7/04; G10L 25/51; H04R 3/04; H03F 2200/03; H03F 3/183; H04R 2400/01; A61B 2562/0204; H04R 29/001

(57) ABSTRACT

An improved method and apparatus for detecting and measuring one or more biometric parameters of a user using a computing device in conjunction with an electroacoustic (audio) transducer is described. A first mode in which the audio transducer produces sound is disabled, and the device is placed in a second mode of operation in which a biometric signal is recovered from the transducer using a “back” audio signal. The biometric signal may then be measured or analyzed. The first mode is disabled by temporarily creating a high impedance between circuitry producing the audio signal and the transducer, while the biometric parameter is measured. This allows for detection of the biometric event without the need for significant additional components or circuitry. The computing device may most conveniently be a smartphone, but the approach described herein may also be easily and usefully applied to tablets, laptop or desktop computers or other devices.

This application claims priority from Provisional Application No. 63/016,012, filed Apr. 27, 2020, which is incorporated by reference herein in its entirety.

### FIELD OF THE INVENTION

The present invention relates generally to electrical devices, and more particularly to the use of audio output devices as input devices for biometric detection data.

### BACKGROUND OF THE INVENTION

Fitness trackers and similar devices have recently become available that are able to detect and measure one or more biometric parameters of a user; for example, some devices can measure a user's heart rate, blood oxygen level, temperature or other parameter. One common example is a FitBit® watch that has the ability to measure a user's heart rate by using light-emitting diode (LED) light output and a photo sensor light input device.

The FitBit® watch uses green LEDs that flash hundreds of times per second and light-sensitive photodiodes to detect from those flashes volume changes in capillaries in the user's wrist. A processor then calculates the number of heartbeats per minute from the volume changes. However, this technique requires both additional output and data acquisition devices, i.e., the LEDs and the light-sensitive photodiodes, as well as the processor.

As with the development of fitness trackers, smartphones have become able to incorporate more functions; user demand for additional features in smartphones drives the desire of the manufacturers of such devices to differentiate themselves from the competition. One possible differentiation is the addition of a biometric measurement such as those provided by the FitBit® watch; a cell phone capable of this additional measurement may be considered likely to be more desirable that one that does not have this feature.

However, the smartphone business is very competitive. Even if output and data acquisition devices such as those used in fitness trackers such as the FitBit® watch can be incorporated into a smartphone to add such a differentiating feature, this will result in increased costs and decreased profitability if the price of the smartphone cannot be increased enough to cover those costs. Further, those manufacturers who make smartwatches that communicate with their smartphones have seen that not every smartphone purchaser will also purchase a related smartwatch.

It would be useful to be able to use a smartphone or other computing device to detect and measure biometric parameters of a user without the need for significant additional components or circuitry.

### SUMMARY OF THE INVENTION

An improved system and method for detecting and measuring biometric parameters of a user with a computing device is disclosed.

One embodiment describes a circuit for using an audio transducer with a computing device to detect a biometric event, comprising: an electroacoustic transducer configured to receive an input signal and produce sound representing the input signal, and to generate a back signal from pressure impinging upon the electroacoustic transducer; a biometric event detection circuit configured to receive the back signal and to convert the back signal from an analog signal to a digital signal; and means for creating a high impedance between a source of the audio signal and the transducer such that when the high impedance is created the transducer does not produce sound representing the input signal.

Another embodiment describes a method of using an audio transducer with a computing device to detect a biometric event, the electroacoustic transducer configured to receive an input signal and produce sound representing the input signal and to generate a back signal from pressure impinging upon the electroacoustic transducer, the method comprising: creating a high impedance between a source of the input signal and the transducer such that when the high impedance is created the transducer does not produce sound input signal; connecting a biometric event detection circuit to the transducer, the biometric event detection circuit configured to receive the back signal and to convert the back signal from an analog signal to a digital signal; and detecting the biometric event by analyzing the digitized back signal.

### BRIEF DESCRIPTION OF THE DRAWINGS

FIG. 1 is a diagram of an audio subsystem that may be used in a computing device according to the prior art.

FIG. 2 is a diagram of an audio subsystem in a computing device for detecting a biometric event according to one embodiment.

FIG. 3 is a flowchart of a method of detecting a biometric event using a computing device having an audio transducer according to one embodiment.

### DETAILED DESCRIPTION OF THE INVENTION

Described herein is a method and apparatus whereby one or more biometric parameters of a user can be detected and measured using a computing device associated with an electroacoustic (audio) transducer, without the need for significant additional components or circuitry, thus providing an additional feature to the smartphone user at no or minimal additional cost. The computing device may most conveniently be a smartphone, but the approach described herein may also be easily and usefully applied to other devices such as tablets, smartwatches, fitness trackers, laptop or desktop computers or other devices having the appropriate components and functions described herein.

In the described system and method, a first mode in which the smartphone produces audio through an audio transducer is disabled, and the smartphone is placed in a second mode of operation in which a means to recover a signal from the transducer using a “back” or “reverse” signal is used. The biometric event must be one that produces a sound or pressure wave to which the transducer can respond. The first mode may be disabled by, for example, temporarily imposing a high impedance between the circuitry producing the audio signal and the audio transducer, while the biometric parameter is measured.

It is well known in the art that any electroacoustic transducer that produces sound in response to an input electrical audio signal (hereafter an “input signal”), such as the transducer in an earpiece or a loudspeaker, does so by moving in response to the input signal applied to it and thus producing a pressure wave which creates sound corresponding to the input signal. The process works in reverse as well; when such a transducer is subjected to an external sound or pressure wave, it in turn produces an electrical signal, although this signal will typically be orders of magnitude smaller than the signal that is used to drive the transducer. (Most transducers operate in response to analog signals; digital audio signals are typically converted into analog signals before being delivered to a transducer.)

This is the same principle as that of a microphone, which produces an electrical signal in response to a sound pressure wave. The signal produced by a transducer, normally used to produce sound, in response to an external sound may be thought of as a “reverse” or “back signal” to differentiate it from the input signal that is normally applied to the transducer to cause it to produce sound. This “microphone effect” of an earpiece or loudspeaker may be used in some instances to provide a signal representative of some biometric parameter of a user.

While there have been other attempts to use the microphone effect of an earpiece or loudspeaker to measure biometric parameters, such efforts typically take place in a “full duplex” environment, i.e., they attempt to detect the biometric parameter while audio is being produced by the transducer. Because the back signal is generally much smaller than the input signal, such attempts typically require significant additional circuitry and processing to separate or extract the relatively small back signal created by the transducer from the much larger input signal being applied to the transducer at the same time. By contrast, the present apparatus and method operates in a half-duplex mode, in which the biometric parameter is detected while audio is not being produced by the transducer.

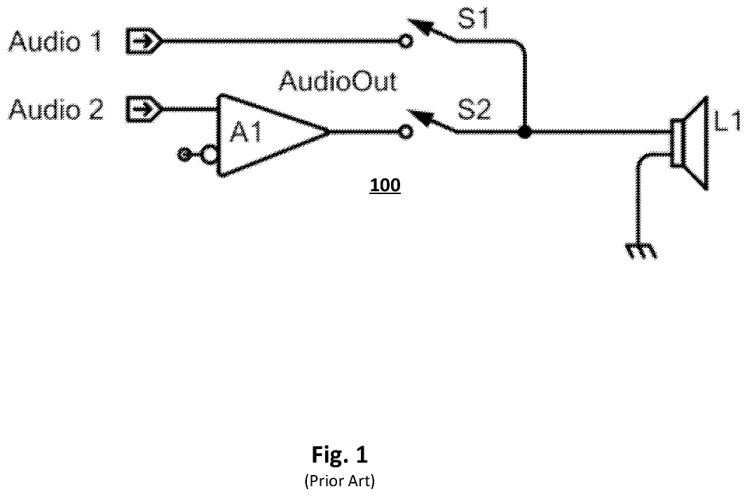

FIG. 1 shows one embodiment of an audio subsystem 100 that might be used in a computing device such as a smartphone, tablet, smartwatch, fitness tracker, laptop or desktop computer or other device according to the prior art. Such a device may contain one or more audio sources; subsystem 100 as illustrated contains two audio sources (not shown), providing two input (audio) signals labeled Audio 1 and Audio 2. The two audio sources may be configured so that only one input signal is produced at a time, or in some cases may be allowed to produce both input signals at the same time.

For example, one audio source may produce an input signal of a telephone call, while the other audio source may produce an input signal of recorded music that is stored on the computing device. An audio source may provide an input signal large enough to drive a transducer without further amplification, such as Audio 1 as illustrated in FIG. 1, or as with signal Audio 2 may require further amplification, here by an amplifier A1, to drive the transducer.

The input signals Audio 1 and Audio 2 are each capable of causing a transducer L1 to convert the input signals into sound that a user can hear. As above, transducer L1 may be one or more loudspeakers, headphones, or one or more earpieces or earbuds.

It is known in the art that a given input signal to an audio transducer may be disabled so that one of multiple signal sources may be selected to drive the transducer to produce sound by causing any given signal source to see a high impedance rather than the load of the transducer. One means of providing such a high impedance is by use of a switch that disconnects a signal source from the transducer. In subsystem 100 switches S1 and S2 are provided as illustrated so that the audio sources, and thus audio signals Audio 1 and Audio 2, may be independently disconnected from transducer L1 when desired; in their open positions, switches S1 and S2 disconnect Audio 1 and Audio 2, respectively, from transducer L1. When one of the audio signals is desired to power transducer L1, the appropriate switch between transducer L1 and that audio source is closed.

Another way in which the output to an audio transducer may be disabled is by means of a so-called “third state” or tri-state amplifier output that is high impedance. For example, U.S. Pat. No. 9,065,384 uses a combination of both switch and tri-state means to select a signal that is fed to the audio output device. High impedance can also be created using reactive components such as capacitors and inductors; in these cases the high impedance is frequency selective, and a circuit can be designed to allow the transducer to see a high impedance in one portion of the frequency spectrum, and a low impedance in anther portion.

In all cases in which either a single signal source or multiple potential signal sources drive the audio device, the known art teaches how each of the signal sources may be separately made to see a high impedance and thus incapable of driving the transducer and those of skill in the art will be able to use these and other methods of creating such a high impedance.

As above, an audio transducer such as transducer L1 in subsystem 100 will produce a back signal in response to sound or pressure not produced by the transducer. Thus, in some instances in which a biometric event creates a sound or other pressure that can be detected by the transducer, the transducer that is normally an audio output device may be used as a biometric detection device.

In the present approach, the first mode of operation, i.e., audio reproduction, is discontinued and a second mode of operation is activated. In this second mode, the input signal to the transducer is disabled or made inoperative, and a means to detect the back signal from the transducer, and recover from the back signal a signal representing the biometric event, is enabled.

Discontinuing the first mode of operation while the biometric event is detected makes it easier to capture and measure the back signal, since the back signal now must only be extracted and distinguished from any electrical or ambient noise, and not from the much larger input signal of the first, normal mode of operation. Further, as will be discussed below, given that the back signal is again typically small, it is helpful if the transducer is closely “coupled” to the biometric event that is to be detected so as to maximize the size of the back signal and make the biometric event easier to detect and measure.

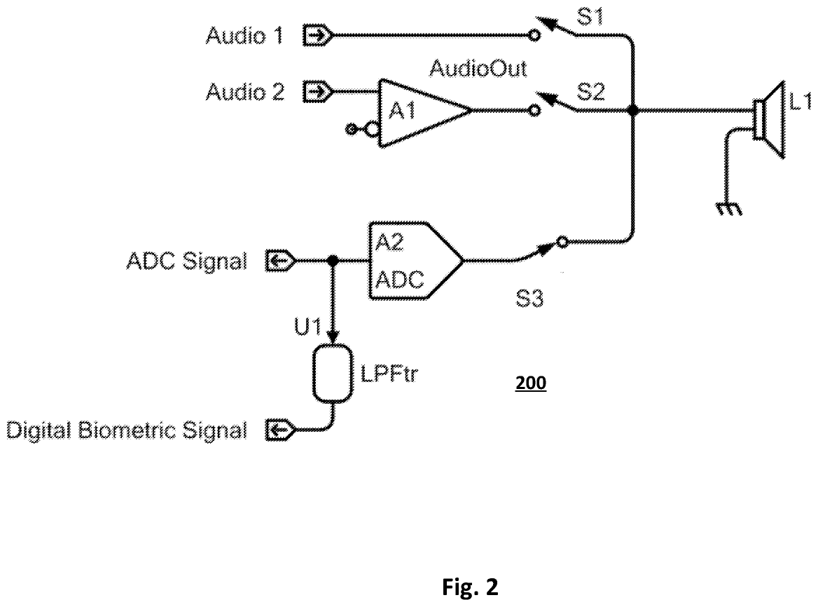

FIG. 2 is a diagram of an audio subsystem 200 for detecting a biometric event that may be used with a computing device such as a smartphone, tablet, smartwatch, fitness tracker, laptop or desktop computer or other device according to one embodiment.

Audio subsystem 200 contains all of the components of audio subsystem 100 of FIG. 1, so that it is able to select via switches S1 and S2 which input signal, again here Audio 1 or Audio 2, is to be fed to transducer L1, and to produce audio from transducer L1, as in the prior art of FIG. 1. In addition, unlike audio subsystem 100, audio subsystem 200 contains components forming a biometric event detection circuit that allows for the recovery of a signal representing a biometric event; however, the number and arrangement of these components is fewer and simpler than those in prior art systems because the back signal need not be detected within, or filtered from, the larger input signal.

The back signal produced by transducer L1 will be a pressure wave signal of some frequency, and will typically be converted to a digital signal using an analog to digital convertor (ADC); in circuit 200 of FIG. 2, an ADC A2 receives the back signal from transducer L1, and converts it into a digital signal here labeled ADC Signal. If the level of the back signal is too small, a programmable gain amplifier (PGA; not shown) may be used to increase the back signal prior to it being input to the ADC A2. While ADC Signal may be analyzed at this point, it may be desirable to filter ADC Signal before analysis.

Since various biometric events are expected to occur at certain, generally low, frequencies, a low-pass filter LPFtr is included in circuit 200 to remove undesirable or unnecessary frequencies from ADC Signal and limit the frequency range to that in which the biometric event is expected to occur. This results in a digital biometric signal that may then be further processed as desired to measure or otherwise categorize the biometric event.

One of skill in the art will appreciate how the digital biometric signal (or ADC Signal, before low pass filtering) may be measured or otherwise analyzed in a variety of ways known in the prior art, and that the analysis performed may depend upon the particular type of biometric event being detected and analyzed. It will also be appreciated that in some cases changes may be desirable to typical processing according to the prior art. For example, in many instances of signal processing, the absolute value of a signal is taken, and then its root mean square (RMS) is calculated and used for further processing and/or measuring, as is often done with, for example, electrocardiogram (ECG) signals. However, in some instances, doing this with the digital biometric signal in a computing device environment may result in interference from, for example, radio stations as some of the circuitry acts like a radio antenna. In this instance, it will be better to just narrow the band in which the signal is expected to occur and process the digital biometric signal as it is without taking its absolute value or RMS. One of skill in the art will be able to determine when this is necessary or appropriate.

In operation of the second mode, when a biometric event is to be detected, both switches S1 and S2 are open (or high impedance is otherwise provided as described above or otherwise known in the art) so that no input signal causes transducer L1 to produce sound. Switch S3, which will normally be open during the playing of audio by transducer L1, is closed as shown in FIG. 2 so that the back signal from transducer L1 is fed to ADC A2.

As a practical matter, switch S3 should be open when either switch S1 or switch S2 is closed, and switches S1 and S2 should be open when switch S3 is closed. The voltage through switch S1 or S2, i.e., the input signal, will typically be on the order of 1 to 2 volts RMS, while the voltage expected through switch S3 is on the order of microvolts (μV).

As above, if an input signal drives transducer L1, any attempt to detect a small back signal from the audio playing will be very difficult. On the other hand, if the audio signal is coming from amplifier A1, for example, and switch S3 is closed so that ADC A2 is not disconnected, amplifier A1 may have performance problems; for example, it may hit a non-linear portion of its operation and clamp or distort. Further, if there is a PGA being used to amplify the back signal, connecting the PGA to the output of amplifier A1 will result in an enormous input to ADC A2, possibly damaging the circuitry.

Because in operation as described herein the transducer L1 does not produce audio output while it is operating to detect the biometric event, the circuit is operating in a “half-duplex” mode in the present approach. This may be distinguished from the “full-duplex” mode of some prior art in which the back signal representing the biometric event must be extracted from the sound being produced by the transducer in response to an input signal.

It is known that certain biometric events result in sound or pressure waves; it is these waves that are being detected by the transducer. In the present approach a relationship has been found between certain back audio signals and particular biometric measurements.

For example, a back signal generated by an earbud, or a pair of earbuds, placed in a user's ear canal has been found to be due to the blood flow in the region of the ear, and thus represents the user's heart rate. Because the effect of the blood flow on the transducer, and thus the back signal, is small, closely coupling the transducer to the ear, by “sealing” the earbuds to the user's ears as much as is practical by being tightly placed in the user's ear canals, will improve the detection of the blood flow and thus the measurement of the heart rate.

Another biometric measurement that may be detected in a similar fashion is a tremor of the user's hand; if the mode of operation for detecting a biometric event is enabled while the user is holding, for example, a smartphone, the movement of the smartphone due to the tremor may cause a low frequency back signal to be generated from the loudspeaker(s) of the smartphone.

As above, the low pass filter LPFtr is configured to pass only those frequencies that are expected from a biometric event; in general, biometric events are expected to occur at low frequencies. For example, heart rates are generally less than 200 beats per minute, or just over 3 hertz (Hz, or cycles per second). For the hand tremor example, a low frequency band should also suffice, as muscle tremors would similarly not be expected to occur much more frequently than a heart beat, if even that fast. Thus, LPFtr may, for example, be set to pass frequencies from 0 to 10 Hz and be expected to capture any of these biometric events. One of skill in the art will be able to determine when and if higher frequency limits may be appropriate.

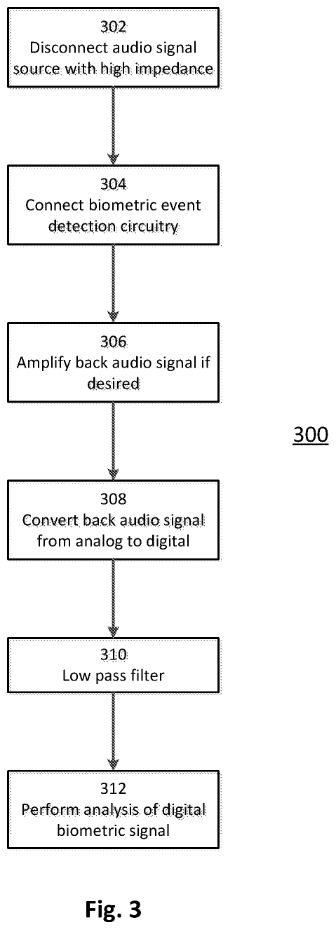

FIG. 3 is a flowchart of a method of detecting and measuring a biometric event using a computing system having an audio transducer according to one embodiment.

At step 302, any audio input signal source in the computing device is disconnected by creating a high impedance between the input signal source and the transducer. This may be accomplished, for example, by opening switches between the audio source(s) and the audio transducer, such as switches S1 and S2 in audio subsystem 200 of FIG. 2, and allows the transducer to produce a back (analog) signal in response to the biometric event without audio being produced by the transducer at the same time.

Next, at step 304, the circuitry to detect the biometric event is connected to the transducer. In audio subsystem 200 of FIG. 2, this is done by closing switch S3 between transducer L1 and ADC A2.

At step 306, the back signal created by the transducer in response to the biometric event is amplified if it is deemed necessary or desirable to do so. One of skill in the art will be able to determine whether such amplification is needed to make the back signal large enough for further processing.

At step 308, the back signal is converted from the analog signal created by the action of the transducer into a digital signal. For example, in audio subsystem 200 of FIG. 2, ADC A2 performs this conversion. This results in a digital signal that represents the biometric event.

At step 310, the digital signal created by the analog to digital conversion is filtered to eliminate frequencies that are not of interest. Since most biometric events take place at low frequencies, a low pass filter such as LPFtr in audio subsystem 200 of FIG. 2 will generally be used to filter the digital signal. This results in a digital biometric signal that represents the biometric event.

Lastly, at step 312, the digital biometric signal is analyzed to measure or otherwise determine parameters of the biometric event. For example, a heart rate may be analyzed to determine whether or not it is regular and/or how fast it is. One of skill in the art will be able to determine what measurements and analysis are available for various biometric events.

As above, in the present approach the circuit has two modes, a first mode in which the transducer produces sound in response to an input signal, and a second mode in which the transducer does not produce sound while it is being used to detect a biometric event. Thus, a circuit as described herein may be considered to operate in a “half-duplex” mode, rather than in the “full-duplex” mode of prior art applications in which audio is produced at the same time the biometric event is detected. One of skill in the art will appreciate that it is possible to switch between the two modes at such a high rate that the system will appear to a user to be operating in full-duplex mode, although it is still operating in a half-duplex mode.

By combining these features, it is possible to construct a device that can detect and measure certain biometric events using a conventional computing device with no significant additional components, as again it is not necessary to filter or otherwise separate the back signal from the input signal as in the prior art. Note that although the present approach is primarily described in the context of a smartphone, it will be clear to those skilled in the art that the approach may be used with other computing devices.

The disclosed system has been explained above with reference to several embodiments. Other embodiments will be apparent to those skilled in the art in light of this disclosure. Certain aspects of the described method and apparatus may readily be implemented using configurations other than those described in the embodiments above, or in conjunction with elements other than or in addition to those described above.

It is to be understood that the examples given are for illustrative purposes only and may be extended to other implementations and embodiments with different conventions and techniques. While a number of embodiments are described, there is no intent to limit the disclosure to the embodiment(s) disclosed herein. On the contrary, the intent is to cover all alternatives, modifications, and equivalents apparent to those familiar with the art.

These and other variations upon the embodiments are intended to be covered by the present disclosure, which is limited only by the appended claims.

What is claimed is:

1. A circuit for using an audio transducer with a computing device to detect a biometric event, comprising:
an electroacoustic transducer configured to receive an input signal and produce sound representing the input signal, and to generate a back signal from pressure impinging upon the electroacoustic transducer;
a biometric event detection circuit configured to receive the back signal and to convert the back signal from an analog signal to a digital signal; and
means for creating a high impedance between a source of the audio signal and the transducer such that when the high impedance is created the transducer does not produce sound representing the input signal.

2. The circuit of claim 1 wherein the means for creating a high impedance is a switch having a first position in which the source of the input signal is connected to the transducer and a second position in which the source of the input signal is disconnected from the transducer.

3. The circuit of claim 1 wherein the means for creating a high impedance is a tri-state amplifier output that is high impedance.

4. The circuit of claim 1 wherein the biometric event detection circuit comprises an analog to digital converter.

5. The circuit of claim 4 wherein the biometric event detection circuit further comprises a low pass filter.

6. The circuit of claim 5 wherein the low pass filter is configured to pass frequencies below approximately 10 hertz.

7. The circuit of claim 1 further comprising a switch having a first position in which the biometric event detection circuit is connected to the transducer and a second position in which the biometric event detection circuit is disconnected from the transducer.

8. The circuit of claim 1 wherein the transducer comprises one or more loudspeakers, headphones or earbuds.

9. The circuit of claim 1 wherein the computing device is a smartphone, tablet, smartwatch, fitness tracker, laptop computer or desktop computer.

10. The circuit of claim 1 further comprising an amplifier located between the transducer and the biometric event detection circuit and configured to amplify the back signal.

11. A method of using an audio transducer with a computing device to detect a biometric event, the electroacoustic transducer configured to receive an input signal and produce sound representing the input signal and to generate a back signal from pressure impinging upon the electroacoustic transducer, the method comprising:
creating a high impedance between a source of the input signal and the transducer such that when the high impedance is created the transducer does not produce sound input signal;
connecting a biometric event detection circuit to the transducer, the biometric event detection circuit configured to receive the back signal and to convert the back signal from an analog signal to a digital signal; and
detecting the biometric event by analyzing the digitized back signal.

12. The method of claim 11 wherein the a high impedance is created by a switch having a first position in which the source of the input signal is connected to the transducer and a second position in which the source of the input signal is disconnected from the transducer.

13. The method of claim 11 wherein the high impedance is created by a tri-state amplifier output that is high impedance.

14. The method of claim 11 wherein the biometric event detection circuit comprises an analog to digital converter.

15. The method of claim 14 wherein the biometric event detection circuit further comprises a low pass filter.

16. The method of claim 15 wherein the low pass filter is configured to pass frequencies below approximately 10 hertz.

17. The method of claim 11 wherein the biometric event detection circuit is connected by a switch having a first position in which the biometric event detection circuit is connected to the transducer and a second position in which the biometric event detection circuit is disconnected from the transducer.

18. The method of claim 11 wherein the transducer comprises one or more loudspeakers, headphones or earbuds.

19. The method of claim 11 wherein the computing device is a smartphone, tablet, smartwatch, fitness tracker, laptop computer or desktop computer.

20. The method of claim 11 further comprising amplifying the back signal from the transducer such that the biometric event detection circuit receives an amplified back signal.
